(19) United States
(12) Patent Application Publication
(10) Pub. No.: US 2023/0000990 A1
(43) Pub. Date: Jan. 5, 2023

(54) NON-VIRAL VECTORS COMPRISING POLYPROPYLENEIMINE

(71) Applicant: UNIVERSITEIT GENT, Gent (BE)

(72) Inventors: Richard HOOGENBOOM, AB Terneuzen (NL); Niek SANDERS, Erpe-Mere (BE)

(73) Assignee: UNIVERSITEIT GENT, Gent (BE)

(21) Appl. No.: 17/784,859

(22) PCT Filed: Dec. 17, 2020
(86) PCT No.: PCT/EP2020/086606

§ 371 Date: Jun. 13, 2022

(30) Foreign Application Priority Data
Dec. 17, 2019 (EP) 19217056.1

(51) Int. Cl. A61K 47/34; C12N 15/87; A61K 31/713; A61K 47/14

(52) U.S. Cl. CPC A61K 47/34; C12N 15/87; A61K 31/713; A61K 47/14

(57) ABSTRACT

The present invention relates to the field of non-viral vectors and pharmaceutical compositions comprising polypropyleneimine and a nucleic acid, and their use in human or veterinary medicine. More precisely, the present invention relates to pharmaceutical compositions comprising a polymer or co-polymer of polypropyleneimine for delivery or transfection of a nucleic acid, e.g. RNA. The pharmaceutical compositions described herein are particularly useful for (nucleic acid) vaccination, nucleic acid-based protein therapy, nucleic-acid based protein replacement therapy, gene editing, base editing, cell therapy, immunotherapy, stem cell therapy, regenerative medicine, gene silencing, nucleic acid inhibition or protein inhibition.

### FIELD OF THE INVENTION

The present invention relates to the field of non-viral vectors and pharmaceutical compositions comprising polypropyleneimine and a nucleic acid, and their use in human or veterinary medicine. More precisely, the present invention relates to pharmaceutical compositions comprising a polymer or co-polymer of polypropyleneimine for delivery or transfection of a nucleic acid, e.g. RNA. The pharmaceutical compositions described herein are particularly useful for (nucleic acid) vaccination, nucleic acid-based protein therapy, nucleic-acid based protein replacement therapy, gene editing, base editing, cell therapy, immunotherapy, stem cell therapy, regenerative medicine, gene silencing, nucleic acid inhibition or protein inhibition.

### BACKGROUND TO THE INVENTION

The introduction of foreign nucleic acids encoding one or more polypeptides for prophylactic and therapeutic purposes has been a goal of biomedical research for many years, especially in light of advancements in gene therapy. Nevertheless, the introduction of foreign nucleic acids has been proved useful more specifically in the context of nucleic based vaccination, protein therapy, protein replacement therapy, gene editing, base editing, cell therapy, immunotherapy, stem cell therapy, regenerative medicine, gene silencing, RNA inhibition or protein inhibition. Nucleic acid delivery is a promising new tool having several applications that could treat some diseases that currently are incurable such as, genetic disorders, cancer diseases and some retinal diseases, and can also be used in vaccination purposes. Nucleic acid delivery consists in the introduction of nucleic acids, such as RNA and DNA, into cells. Since naked nucleic acids as such are typically not efficiently internalized by cells, a carrier system (vector) is needed for nucleic acid delivery. The introduction of foreign nucleic acids in cells varies in light of the target cell or organism, the type of nucleic-acid molecule and/or the delivery system. Influenced by safety and efficacy concerns associated with the use of deoxyribonucleic acid (DNA) molecules, ribonucleic acid (RNA) molecules have received growing attention in the recent years. Various approaches have been proposed for the delivery of RNA, e.g. non-viral or viral delivery vehicles. In viruses and in viral delivery vehicles, the nucleic acid is typically encapsulated by proteins and/or lipids (virus particle). For example, engineered RNA virus particles derived from RNA viruses have been proposed as delivery vehicle for treating plants or for vaccination of mammals. A variety of compounds for the vectorization of nucleic acids, so-called transfection reagents, have been described previously. These compounds are usually either polycations or compositions comprising cationic lipids or lipid-like compounds such as lipidoids. Complexes of nucleic acids with polycations are referred to as polyplexes, those with cationic lipids are referred to as lipoplexes.

While viruses are the most efficient delivery vehicles currently available, their possible use raised safety concerns. The medical and veterinary community is reluctant to administer RNA virus particles to humans or animals. For all the above-mentioned reasons, other types of vectors, which do not comprise virus particles, are currently investigated. Non-viral vectors currently investigated comprise polymers, which have been found advantageous due to their chemical flexibility, ease of synthesis, potential for biocompatibility, simplicity, and inexpensive synthesis.

Prior art discloses the use of polymers such as PEI and PGA for the delivery of biomacromolecules (WO2018/156617 A2). The use of polymer micelles for the delivery of various therapeutic drugs has also been described (WO2018/002382 A1). However, the compositions in the prior art often demonstrate shortcomings such as low transfection efficiency or are limited by their cytotoxicity. Therefore, even though non-viral vectors have been extensively investigated in the context of nucleic acid delivery, the translation of non-viral vector approaches into clinical practice has not been very successful for various reasons, i.e. toxicity, unsatisfying transfection efficiency, technological and regulatory problems. Thus, there is a need for alternative pharmaceutical compositions for delivery and transfection of nucleic acids. In the present invention, we have identified novel non-viral vectors that are efficient in nucleic delivery and transfection, and which overcome the above defined issues. These vectors are characterized in comprising PPI, preferably having a low degree of polymerization, more preferably being linear PPI.

A specific finding of the present invention, is that L-PPI monomers and L-PPI/L-PEI co-polymers, having a high PPI content were found to more efficiently complex RNA compared to L-PEI monomers and L-PPI/L-PEI polymers, having a low PPI content.

### SUMMARY OF THE INVENTION

In a first aspect, the present invention provides for a novel composition comprising a polypropyleneimine polymer (PPI) and a nucleic acid. More specifically, the present invention provides for a pharmaceutical composition comprising PPI and a nucleic acid, and wherein said PPI has a degree of polymerization from about 20 to 1000, preferably from about 100 to 500, most preferably from about 200 to 300.

In a preferred embodiment, said PPI is linear.

In a further embodiment, the composition further comprises a polyethyleneimine polymer (PEI). In another embodiment, said PEI is linear.

In a further embodiment, said PEI has a degree of polymerization from about 20 to 1000, preferably from about 100 to 500, most preferably from about 200 to 300.

In a particular embodiment, said PPI and said PEI form a co-polymer, which can be a random co-polymer. Accordingly, the present invention also provides a pharmaceutical composition comprising a PPI/PEI co-polymer.

In a particular embodiment in accordance with the present invention, said co-polymer has a degree of polymerization from about 20 to 1000, preferably from about 100 to 500, most preferably from about 200 to 300.

In a particular embodiment, the degree of polymerization of said PEI to the degree of polymerization of said PPI in the compositions or co-polymers of the present invention is within a range from about 1:1 to 1:500, preferably from about 1:1 to 1:100, most preferably from about 1:2 to 1:10.

In one embodiment, the pharmaceutical composition further comprises lipids.

In yet another embodiment, the nucleic acid is an RNA or DNA molecule; preferably selected from the list comprising mRNA, self-replicating mRNA (replicon), circular mRNA, circular RNA, a mRNA or replicon whose translation can be controlled by an external or internal molecule, non-coding RNA, siRNA, sense RNA, antisense RNA, a ribozyme, an RNA aptamer, an RNA aptazyme, saRNA, pDNA, mini circles, closed linear DNA, genomic DNA, cDNA, either single- and/or double-stranded DNA, and any combination or chemical modified version thereof.

In yet a further embodiment, the N/P ratio is less than 40; preferably less than 20; more preferably less than 10.

In another embodiment, the pharmaceutical composition according to the present invention is for use in human or veterinary medicine, more specifically, the pharmaceutical composition is for use in (nucleic acid) vaccination, nucleic acid-based protein therapy, nucleic-acid based protein replacement therapy, gene editing, base editing, cell therapy, immunotherapy, stem cell therapy, regenerative medicine, gene silencing, nucleic acid inhibition or protein inhibition.

An advantage of the present invention is that the composition has high transfection efficiency, a low cytotoxicity compared to state-of-the-art non-viral carriers and a further advantage is that their small size renders them good for in vivo use.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1, also abbreviated as FIG. 1, illustrates the transfection efficiency of a composition comprising linear PPI (L-PPI) and a DP of 250 in accordance with the present invention,

FIG. 2, also abbreviated as FIG. 2, illustrates the transfection efficiency of a composition comprising linear PEI (L-PEI) and a DP of 250.

FIG. 3, also abbreviated as FIG. 3, illustrates the transfection efficiency of a composition comprising a co-polymer of linear PEI and linear PPI (L-PEI/L-PPI) having DP 50/200.

FIG. 4, also abbreviated as FIG. 4, illustrates the transfection efficiency of a composition comprising a co-polymer of linear PEI and linear PPI (L-PEI/L-PPI) having DP 200/50.

FIG. 5, also abbreviated as FIG. 5, illustrates the transfection efficiency of a composition comprising a co-polymer of linear PEI and linear PPI (L-PEI/L-PPI) having DP 100/150.

FIG. 6, also abbreviated as FIG. 6, illustrates the in vitro transfection efficiency of a composition comprising a co-polymer of linear PEI and linear PPI (L-PEI/L-PPI) having DP 150/100.

FIG. 7, also abbreviated as FIG. 7, illustrates the transfection efficiency of a composition comprising modified mRNA and linear PEI (L-PEI) having a DP of 250.

FIG. 8, also abbreviated as FIG. 8, illustrates the gene silencing efficacy in HeLa cells of a composition comprising siRNA and a co-polymer of linear PEI and linear PPI (L-PEI/L-PPI) having a DP 50/200.

FIG. 9, also abbreviated as FIG. 9, illustrates the gene silencing efficacy in SKOV3-Luc cells of a composition comprising siRNA and a co-polymer of linear PEI and linear PPI (L-PEI/L-PPI) having a DP 50/200.

FIG. 10, also abbreviated as FIG. 10, illustrates measures of Z potential of a composition comprising linear PPI (L-PPI) having DP 250.

FIG. 11, also abbreviated as FIG. 11, illustrates measures of Z potential of a composition comprising a co-polymer of linear PEI and linear PPI (L-PEI/L-PPI) having DP 50/200.

FIG. 12, also abbreviated as FIG. 12, illustrates size measurements of a composition comprising linear PPI (L-PPI) having DP 250, and replicon RNA.

FIG. 13, also abbreviated as FIG. 13, illustrates size measurements of compositions comprising linear PPi (L-PPi) having DP 250, and modified non-replicating mRNA.

FIG. 14, also abbreviated as FIG. 14, illustrates size measurements of a composition comprising a co-polymer of linear PEI and linear PPI (L-PEI/L-PPI) having DP 50/200, and replicon RNA.

FIG. 15, also abbreviated as FIG. 15, illustrates the cell availability of a composition comprising linear PPI having DP 250.

FIG. 16, also abbreviated as FIG. 16, illustrates the cell availability of a composition comprising linear PEI having DP 250.

FIG. 17, also abbreviated as FIG. 17, illustrates the cell availability of a composition comprising a co-polymer of linear PEI and linear PPI (L-PEI/L-PPI) having DP 50/200 (ratio of 1:4).

FIG. 18, also abbreviated as FIG. 18, illustrates the cell availability if a composition comprising a co-polymer of linear PEI and linear PPI (L-PEI/L-PPI) having DP 200/50 (ratio of 4:1).

FIG. 19, also abbreviated as FIG. 19, illustrates the cell availability of a composition comprising a co-polymer of linear PEI and linear PPI (L-PEI/L-PPI) having DP 100/150 (ratio of 2:3).

FIG. 20, also abbreviated as FIG. 20, illustrates the cell availability of a composition comprising a co-polymer of linear PEI and linear PPI (L-PEI/L-PPI) having DP 150/100 (ratio of 3:2).

FIG. 21, also abbreviated as FIG. 21, illustrates transfection results of in vitro tests of transfection efficiency carried out with lipofectamine MessengerMax (MM), a state-of-the-art transfection agent.

FIG. 22, also abbreviated as FIG. 22, illustrates the cell availability of a composition comprising a lipofectamine MessengerMax (MM) at a ratio of 2:1 (μl MM:μg mRNA).

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

When describing the compounds of the invention, the terms used are to be construed in accordance with the following definitions, unless a context dictates otherwise.

The present invention describes a pharmaceutical composition comprising: (a) a polyethyleneimine polymer PPI; and (b) a nucleic acid, and wherein said PPI has a degree of polymerization from about 20 to 1000, preferably from about 100 to 500, most preferably from about 200 to 300. We have found that compositions according to the present invention have higher transfection efficiency than other non-viral carriers currently available. Moreover, said compositions have small particle size that renders them proper for in vivo use. Therefore, an advantage of the present invention is that the composition has high transfection efficiency, and a further advantage is that their small size renders them good for in vivo use. Moreover, said compositions induce less cytotoxic effect than compositions based on state-of-the-art non-viral carriers.

The pharmaceutical compositions according to the present invention can be or comprise polymeric blends, co-polymers, homopolymers, block co-polymers, gradient co-polymers and random co-polymers. The pharmaceutical composition can further comprise active pharmaceutical ingredients, and other excipients.

The term “nucleic acid” refers to biomolecules composed by a 5-carbon sugar, a phosphate group and a nitrogenous base. The term nucleic acid comprises DNA and RNA, either single- and/or double-stranded, and any combination or chemical modified version thereof.

The term ‘degree of polymerization’, or “DP”, as used herein, unless indicated otherwise, refers to the number-averaged degree of polymerization. It can be calculated using the equation: Mn/M0, where Mn is the number-averaged molecular weight of the polymer and M0 is the molecular weight of the monomer unit. For example, PPI is composed by repeating propylamine units, and therefore propylamine is the monomer unit of PPI. Said monomer unit of PPI has a molecular weight of approximately 57.1 g/mol. Based on the formula above, the number-average molecular weight of a polymer of PPI having DP200 can be calculated as being equal to approximately 11.400 g/mol in its free base form. The polymers can also consist of the protonated form, where the mass of the repeat unit increases with the mass of the salt, e.g. for the HCl salt the mass of the PPI-HCl repeat unit is approximately 93.6 g/mol.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

As used in the specification and the appended claims, the singular forms “a”, “an”, and “the” include plural referents unless the context clearly dictates otherwise. By way of example, “a compound” means one compound or more than one compound.

In accordance with a specific embodiment of the present invention, the composition may further also comprise a polyethyleneimine polymer (PEI).

Polyethyleneimine (PEI) and polypropyleneimine (PPI) are organic macromolecule with a high cationic-charge-density. Polyethyleneimine, also referred to as PEI or poly(ethylene imine), is a polymer composed of repeating ethylamine units. Polypropyleneimine, also referred to as PPI, or poly(propylene imine), is a polymer composed of repeating n-propylamine units. By virtue of their potential to become protonated in view of the presence of charged aminogroups, specifically in their linear form, polymeric compositions comprising PEI and/or PPI can bind and compact nucleic acids. PEI and PPI may compact nucleic acids into positively charged particles capable of interacting with anionic proteoglycans at the cell surface and facilitating entry of the particles.

In accordance with a specific embodiment of the present invention, either PPI or PEI or both, are linear. In the present application linear PPI is also referred to as L-PPI, and linear PEI is also referred to as L-PEI. An advantage of using linear PPI and/or linear PEI is that the resulting pharmaceutical compositions can be positively charged and the formed complexes with nucleic acids have a small size. Another advantage of the polymers is that they are short and hence will be easier cleared by the kidneys.

The L-PPI monomers and L-PPI/L-PEI co-polymers, having a high PPI content were moreover found to more efficiently complex RNA than L-PEI monomers and L-PPI/L-PEI polymers, having a low PPI content.

In accordance with a specific embodiment of the present invention, said PEI has a degree of polymerization from about 20 to 1000, preferably from about 100 to 500, most preferably from about 200 to 300.

The term “average diameter” refers to the mean hydrodynamic diameter of the particles as measured by dynamic light scattering with data analysis using the so-called cumulant algorithm, which provides as results the so-called “Z average” with the dimension of a length. Here “average diameter”, “diameter” or “size” for particles is used synonymously with this value of the Z average.

According to the present invention, “N/P ratio” refers to the molar ratio of nitrogen atoms (N) in the polymer to phosphor atoms (P) in the nucleic acid. The N/P ratio reflects the input molar ratio of nitrogen in a given quantity of polymer to phosphate in a given quantity of nucleic acid. In a specific embodiment, said N/P ratio is less than 40; preferably less than 20; more preferably less than 10, such as for example 5, 4, 3, 2, 1 or less, e.g. 0.5, or 0.2. In a specific embodiment, the N/P ratio may for example be about and between 0.2 and 10; such as between 1 and 10, or between 1 and 5. Alternatively, said ratio may also be between 1 and 20. Specifically, a lower N/P ratio may be beneficial in reducing toxicity of the used compositions. This may for example be the case in compositions having a relatively high PPI content.

In accordance with a further specific embodiment of the present invention, said PPI and said PEI, or said L-PPI and L-PEI, form a co-polymer, preferably a random co-polymer. A co-polymer of L-PPI and L-PEI may for example be represented as follows:

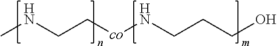

The inventors have surprisingly found that RNA is efficiently complexed and transfected to cells when the co-polymer has preferably a degree of polymerization from about 20 to 1000, more preferably from about 100 to 500, most preferably from about 200 to 300. More specifically, a higher RNA complexing can be achieved in co-polymers having a relatively high ratio of L-PPI to L-PEI.

Particularly preferred compositions of the present invention are characterized in comprising one or more of the following:
a PPE/PEI co-polymer having high PPI content
a PPI having a DP of 250
a PPI/PEI co-polymer having a DP of 250
a PPI/PEI co-polymer having a PPI/PEI ratio of at least 1.5:1, preferably at least 4:1
a N/P ratio of above 1; preferably above 5; more preferably between 5 and 20

A particularly preferred composition of the present invention comprises:
L-PPI having a DP of 250, and
RNA, at an N/P ratio of about between 0.2 and 10; preferably between 1 and 10; most preferably between 1 and 5.

These compositions are particularly characterized in having a high transfection efficiency, a low particle size, a good stability and a low toxicity.

Another particularly preferred composition of the present invention comprises:
a co-polymer of L-PEI and L-PPI, having a DP of 250
RNA, at an N/P ratio of about between 1 and 20
A PPI/PEI ratio of at least 1.5:1; preferably at least 4:1

These compositions are particularly characterized in having a high transfection efficiency, a low particle size, a good stability and a low toxicity.

The term “random co-polymer” as used herein refers to a statistical co-polymer in which the probability of finding a given type monomer residue at a particular point in the chain is similar to the mole fraction of that monomer residue in the chain. This is typically described by the reactivity ratios for the statistical co-polymerization of the parent polymer that is used as precursor for the L-PEI/PPI. Here, we defined a random co-polymer as a co-polymerization for which the reactivity ratios (r₁=k_{p1,1}/k_{p1,2}; r₂=k_{p2,2}/k_{p2,1} with monomer 1 being more reactive) r₁<1.35 and r₂>0.7. The co-polymers of the present invention may contain other polymers besides PPI and/or PEI.

In the context of the present invention, co-polymers of L-PEI and L-PPI having a different molar ratio of L-PEI to L-PPI were synthesized and tested. In accordance with an embodiment of the present invention, the degree of polymerization (DP) of said PEI to the degree of polymerization (DP) of said PPI is within a range from 1:1 to 1:500, preferably from about 1:1 to 1:100, most preferably from about 1:2 to 1:10. In accordance with a specific embodiment of the present invention, it has been found that compositions comprising PEI and PPI which are rich in PPI, show higher transfection efficiencies than other nucleic acid vectors. Compositions rich in PPI are compositions in which the amount of PPI exceeds the amount of any other polymeric component (such as PEI) in the composition. In accordance with the present invention, said nucleic acid is an RNA or DNA molecule; preferably selected from the list comprising mRNA, self-replicating mRNA (replicon), circular mRNA, circular RNA, a mRNA or replicon whose translation can be controlled by an external or internal molecule, non-coding RNA, siRNA, sense RNA, antisense RNA, a ribozyme, an RNA aptamer, an RNA aptazyme, saRNA, pDNA, mini circles, closed linear DNA, genomic DNA, cDNA, either single- and/or double-stranded DNA, and any combination or chemical modified version thereof.

The term “RNA” refers to a molecule which comprises ribonucleotide residues and preferably being entirely or substantially composed of ribonucleotide residues and comprises all RNA types described herein. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally-occurring RNAs. The RNA used according to the present invention may have a known composition, or the composition of the RNA may be partially or entirely unknown.

The term “DNA” refers to a molecule which comprises deoxyribonucleotide residues and preferably being entirely or substantially composed of deoxyribonucleotide residues and comprises all DNA types described herein. The term “DNA” comprises pDNA, mini circles, closed linear DNA, genomic DNA, cDNA, either single- and/or double-stranded DNA, and any combination or chemical modified version thereof.

In one embodiment, the pharmaceutical composition further comprises a lipid. In order to further improve the properties of the pharmaceutical compositions in accordance with the present invention, coformulations with lipids and/or a negatively charged polymer coating can be realized.

The term “lipid” refers to a fatty substance that is insoluble in water and include fats, oils, waxes, and related compounds. Lipids may be either made in the blood (endogenous) or ingested in the diet (exogenous). Lipids are essential for normal body function and whether produced from an exogenous or endogenous source, they must be transported and then released for use by the cells. The production, transportation and release of lipids for use by the cells is referred to as lipid metabolism. While there are several classes of lipids, two major classes are cholesterol and triglycerides. Cholesterol may be ingested in the diet and manufactured by the cells of most organs and tissues in the body, primarily in the liver. Cholesterol can be found in its free form or, more often, combined with fatty acids as what is called cholesterol esters.

The pharmaceutical compositions in accordance with the present invention can be for use in human or veterinary medicine. According to a further embodiment, the pharmaceutical composition in accordance with the present invention may be used in methods in which nucleic acid delivery is useful, such as but not limited to (nucleic acid) vaccination, or nucleic acid-based protein therapy, nucleic-acid based protein replacement therapy, gene editing, base editing, cell therapy, immunotherapy, stem cell therapy, regenerative medicine, gene silencing, nucleic acid inhibition or protein inhibition.

### EXAMPLES

Material and Methods

Synthetic mRNA Production

Luciferase-coding self-amplifying RNAs or replicons derived from Venezuelan Equine Encephalitis Virus (VEEV) were synthesized by in vitro transcription (IVT) using the MEGAscript® kit (Thermo Fisher Scientific, Massachusetts, US). An I-Scel linearized plasmid was used as template. After purification using silica-based columns (RNeasy Mini Kit, Qiagen, Hilden, Germany), the RNA was capped using the ScriptCap™ Cap 1 Capping System Kit (Cellscript, Wisconsin, US) according to the manufacturer's instructions. Finally, the RNA was purified again using silica-based columns and the concentration was determined spectrophotometrically (Nanodrop, Thermo Fisher Scientific, Massachusetts, US). N1-methylpseudouridine (1 mΨ) modified non-replicating mRNAs (mod-mRNA) encoding luciferase were produced by IVT from a I-Scel linearized plasmid by replacing all uridine-5′-triphosphates in the IVT mix by N1-methylpseudouridine-5′-triphosphates (Trilink Biotechnologies, San Diego, USA). Next, the mRNAs were purified and capped using vaccinia virus capping enzymes and 2′-O-methyltransferase (Cellscript, Wisconsin, USA) to create cap1 and were then again purified using the RNeasy mini kit (Qiagen, Germany). The poly(A) tail of these mod-mRNAs, which is 40 adenosines long, was extended using the A-plus Poly(A) polymerase tailing kit (Cellscript) to approximately 200 adenosines, followed by purification. Finally, the mod-mRNA concentration was determined spectrophotometrically (Nanodrop, Thermo Fisher Scientific, Massachusetts, US).

Small Interfering RNAs

Small interfering RNA (siRNA) targeting firefly luciferase (pGL3) or control siRNAs were purchased form Dharmacon (Lafayette, USA) and dissolved in RNase-free water at a concentration of 16.5 μM and stored at −20° C. in aliquots of 20 μl.

Polymer Production

The polymers L-PEI DP 250, L-PPI DP 250 and their co-polymers (L-PEI/L-PPI DP 200/50-150/100-100/150-50/200) were synthesized as follows. First, a co-polymer of 2-ethyl-2-oxazoline (EtOx) and 2-isopropyl-2-oxazine (iPrOzi) (different ratios of the monomers (EtOx:iPrOzi=250:0, 0:250, 200:50, 150:100, 100:150 and 50:200) to make the different co-polymers) was prepared at 4 M total monomer concentration using methyl tosylate as initiator with a total monomer to initiator ratio of 250 to obtain polymers with a DP of 250. The polymerizations were performed in a Biotage microwave reactor at 140° C. to full monomer conversion as confirmed by gas chromatography. Size exclusion chromatography confirmed the formation of rather defined co-polymers with dispersity below 1.4 and ¹H NMR spectroscopy revealed that the targeted compositions were obtained. Subsequently, these copoly(2-oxazoline)s were hydrolysed to obtain the L-PPI and L-PEI/PPI polymers by dissolving 1 gram of polymer in 7.5 mL demi water and 7.5 mL hydrochloric acid (HCl). Then the closed vials were heated up to 140° C. for 9 hours in a Biotage microwave for the hydrolysis. After this the polymer was diluted with demi water and the HCl and demi water were evaporated with reduced pressure. The samples were neutralized with a 2M sodiumhydroxide (NaOH) solution in water and freeze dried. ¹H NMR analysis confirmed near quantitative hydrolysis.

Preparation and Characterization of Self-Amplifying and Modified mRNA Nanocomplexes

To prepare the nanocomplexes, an equal volume of RNA solution was added to the polymer solution and gentle mixed and incubated for 30 minutes at room temperature. Both the polymer and RNA were dissolved in a 20 mM sodium acetate buffer (pH=5.2). Different polymer to mRNA ratios were used to produce nanocomplexes. The N/P ratios were N/P 40-20-10-5-1 and 0.2 for self-amplifying mRNA nanocomplexes, and N/P 30-15-8-4-0.8-0.2 for mod-mRNA nanocomplexes. The size and zeta potentials of the self-amplifying and modified mRNA nanocomplexes were subsequently determined using dynamic light scattering (Zetasizer Nano, Malvern Instruments, Malvern, UK). The zeta potential is a typical measure for the surface charge and hence stability of charged particles in suspension. Typically, a zeta potential of at least circa 20 indicates a good stability of said particles.

Cell Culture and Transfection Procedure

HeLa-cells were cultivated in medium and maintained in a humidified incubator at 37° C. and 5% CO2. The medium consisted of Dulbecco's Modified Eagle Medium (DMEM) (Gibco, Thermo Fisher Scientific, Massachusetts, US) supplemented with 10% Fetal Bovine Serum (Biowest, California, US), 5.000 units/mL penicillin and 5.000 μg/mL streptomycine (Thermo Fisher Scientific, Massachusetts, US). One day before transfection with the self-amplifying or modified mRNAs, the HeLa cells were seeded in 24-well plates at a density of 50000 cells/well. The next day (i.e. 24 h later) the medium was changed to opti-MEM and 20 μL polymer:RNA complex solution, containing 500 ng RNA, was added to each well. Twenty-four hours after transfection, luciferase expression was analyzed by bioluminescence imaging. To that end, Cells were trypsinized and a part of the neutralized cell suspension (60%) was transferred to a black 96-well plate. A D-luciferin solution (50 mg/ml; 10% of final well volume) was added to each well and left to incubate for 10 minutes. Subsequently, the emitted bioluminescent light was measured using the IVIS lumina II (Xenogen Corporation, Alameda, Calif., US). Transfections with the reference carrier Lipofectamine MessengerMax (ThermoFischer Scientific) at different ratios was performed in a similar way using 500 ng RNA per 24 well.

Preparation and In Vitro Silencing Efficacy of siRNA Nanocomplexes

To evaluate the capacity of the polymers to deliver siRNA siRNA-nanocomplexes were prepared by adding an equal volume of siRNA solution was added to the polymer solution and gentle mixed and incubated for 30 minutes at room temperature. Both the polymer and siRNA were dissolved in a 20 mM sodium acetate buffer (pH=5.2). The final concentration of the siRNA was 10 nM. Different polymer (PEI/PPI DP 50/200) to siRNA ratios were used to produce the siRNA-nanocomplexes. Subsequently, these siRNA-nanocomplexes were tested in two protocols.

In a first set of experiments we performed co-transfections of a luciferase replicon with the PEI/PPI siRNA-nanocomplexes in HeLa cells. HeLa were cultivated as described above. One day before co-transfection with the PEI/PPI (50/200) based replicon mRNA- and siRNA-nanocomplexes, the HeLa cells were seeded in 24-well plates at a density of 50,000 cells/well. The next day (i.e. 24 h later) the medium was changed to Opti-MEM and the cells were transfected with 500 ng luciferase encoding replicon using PEI/PPI (DP50/200) at a N/P of 5. After 30 min PEI/PPI siRNA-nanocomplexes containing 6 pmol siRNA were added to the cells. Twenty-four later we measured the luciferase using the IVIS lumina II imaging system as described above for the mRNAs. As controls we used cells that were treated with only the luciferase replicon or with luciferase replicon plus a PEI/PPI nanocomplex containing a scrambled siRNA made at the highest studied N/P ratio. The latter ratio is expected to have the highest cytotoxicity.

In a second set of experiments SKOV3-Luc cells, stabling expression firefly luciferase, were used. These cells were cultivated in a humidified incubator at 37° C. and 5% CO₂ with McCoy's 5A (Modified) Medium (Gibco, Thermo Fisher Scientific, Massachusetts, US) supplemented with 10% Fetal Bovine Serum (Biowest, California, US), 5.000 units/mL penicillin and 5.000 μg/mL streptomycine (Thermo Fisher Scientific, Massachusetts, US). One day before transfection with the PEI/PPI (50/200) based siRNA-nanocomplexes, the SKOV-3-Luc cells were seeded in 24-well plates at a density of 50,000 cells/well. The next day (i.e. 24 h later) the medium was changed to opti-MEM and 20 μL PEI/PPI (50/200) polymer siRNA-nanocomplex solution, containing 6 pmol siRNA, was added to each well. Thirty-six hours after transfection, luciferase expression was analyzed using the IVIS lumina II imaging system as described above for the mRNAs.

Cell Viability

The cell viability after 24 h of transfection experiments was determined using the Cell Proliferation Reagent WST-1 (Roche). After trypsinization, a part of the neutralized volume (6.66%) was transferred to a clear ELISA plate and WST-1 solution was added according to the manufacturer's instructions. After 30 minutes of incubation, the plate was shaken for 1 minute on a plate shaker and the absorbance at 450 nm (620 nm reference) was determined using the EZ Read 400 microplate reader (Biochrom).

In Vivo Transfection

The in vivo transfection efficacy of nanocomplexes comprising self-amplifying mRNA and L-PEI/PPI polymers (DP 50/200) was studied in chickens after local injection in the neck or wing. To that end self-amplifying mRNA-PEI-PPI nanocomplexes were prepared at a N/P ratio of 5 as described for the in vitro experiments. Nanocomplexes containing 5 μg self-amplifying mRNA (encoding luciferase) were subsequently injected in the neck or wing. After two days the chickens were injected with D-luciferin and subsequently euthanized and imaged with IVIS lumina II.

Results

Measures of Transfection Efficiency

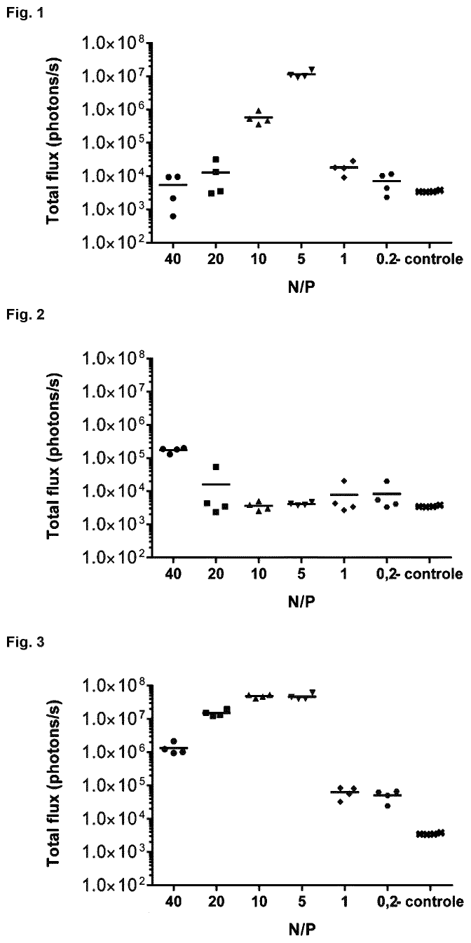

FIGS. 1 to 7 illustrate the results of in vitro tests of transfection efficiency carried out with self-amplifying mRNA nanocomplexes or modified mRNA nanocomplexes. Compositions comprising polymers and nucleic acids with different N/P ratios were prepared. More specifically, 0.2, 1, 5, 10, 20 and 40. A control solution with only buffer and thus containing no nanocomplexes was also prepared (controle, ctrl).

FIG. 1 illustrates transfection results for compositions comprising self-amplifying mRNA and linear PPI (L-PPI) having DP 250, indicating that in each instance the transfection efficiency is increased compared to the control. A particularly increased transfection efficiency is achieved for compositions having an N/P ratio of between 1 and 10; specifically between 5 and 10.

FIG. 2 illustrates transfection results for compositions comprising self-amplifying mRNA and linear PEI (L-PEI) having DP 250. It is important to note that the bioluminescence intensity for the first composition for N/P 10 and N/P 5, and therefore the transfection efficiency, remarkably exceeds the fluorescence intensity of the second composition. In contrast to the results obtained for PPI, for PEI no increased transfection efficiency is observed for the different compositions compared to the control, except for N/P 40.

FIG. 3 illustrates transfection results for compositions comprising self-amplifying mRNA and a co-polymer of linear PEI and linear PPI (L-PEI/L-PPI) having DP 50/200. Therefore, the degree of polymerization of said PEI to the degree of polymerization of said PPI is in a ratio of 1:4. Again an increased transfection efficiency is observed for all compositions compared to the control, with a particularly increased transfection efficiency for compositions having an N/P ratio of above 5.

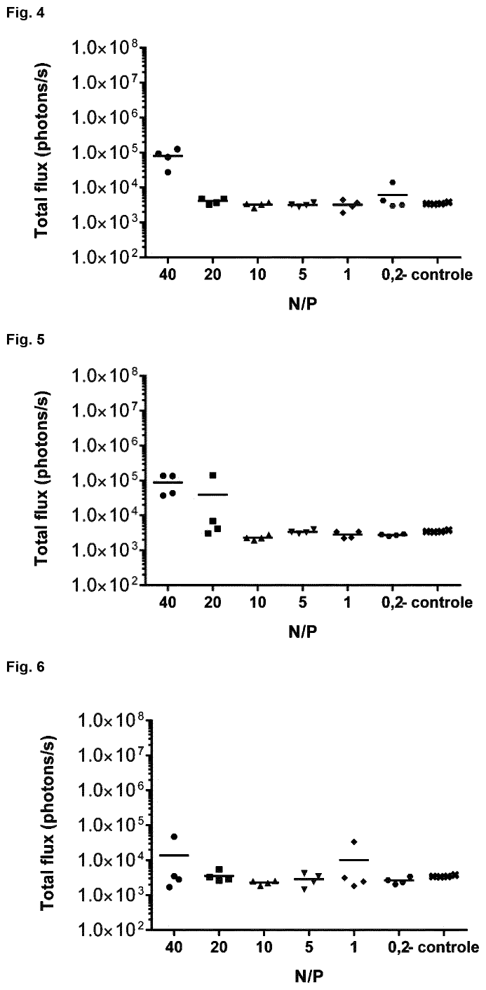

FIG. 4 illustrates transfection results for compositions comprising self-amplifying mRNA and a co-polymer of linear PEI and linear PPI (L-PEI/L-PPI) having DP 200/50 (ratio of 4:1). It is important to note that the bioluminescence intensity of the first composition, which is rich in PPI, remarkably exceeds the bioluminescence intensity of the second composition for each of the N/P ratios tested. Therefore, the first composition rich in L-PPI shows even higher transfection efficiency than the composition comprising L-PPI but not L-PEI (which results are illustrated in FIG. 1, left side).

FIG. 5 illustrates transfection results for compositions comprising self-amplifying mRNA and a co-polymer of linear PEI and linear PPI (L-PEI/L-PPI) having DP 100/150 (ratio of 2:3). This figure shows again that an excess of PPI has a beneficial effect on the transfection efficiency of the tested compositions.

FIG. 6 illustrates transfection results for compositions comprising self-amplifying mRNA a co-polymer of linear PEI and linear PPI (L-PEI/L-PPI) having DP 150/100 (ratio of 3:2). This figure confirms that an excess of PEI in the compositions, does not substantially affect the transfection efficiency of the tested compositions.

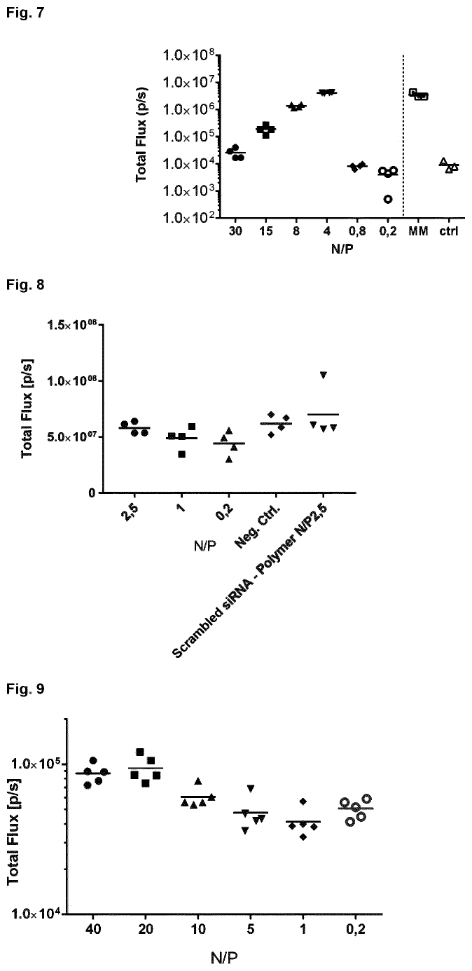

FIG. 7 illustrates transfection results for compositions comprising modified mRNA and linear PPI (L-PPI) having DP 250, indicating that the transfection efficiency is increased compared to the control (ctrl) between an N/P of 0,8 and 30. A particularly increased transfection efficiency is achieved for compositions having an N/P ratio of between 1 and 10. The graph also illustrates in vitro transfection results carried out with lipofectamine MessengerMax (MM), a state-of-the-art transfection agent. Compositions comprising this lipid carrier and modified mRNA (at a ratio of 2 μl MM:1 μg mod-mRNA) typically resulted in transfection efficiencies between 1×10⁶ and 1×10⁷. When using modified non-replicating mRNA we can conclude that the compositions of the present invention are at least equally efficient as MM.

FIG. 8 illustrates transfection results in HeLa cells for compositions comprising siRNA and a co-polymer of linear PEI and linear PPI (L-PEI/L-PPI) having DP 50/200, indicating that the siRNA mediated silencing is most efficient between an N/P of 1 and 0.2 when compared to the scrambled siRNA or HeLa cell that only received the luciferase encoding replicon (neg. Ctrl.) The data were obtained by adding siRNA-nanocomplexes to HeLa cells that were co-transfected with a luciferase encoding replicon. A lower expression (total flux) indicates a good intracellular delivery of the siRNA and subsequent signalling of the target luciferase mRNA.

FIG. 9 illustrates transfection results in SKOV-3-Luc cells for compositions comprising siRNA and a co-polymer of linear PEI and linear PPI (L-PEI/L-PPI) having DP 50/200, indicating that the siRNA mediated silencing is most efficient at N/P ratio of 5 or lower. The SKOV-3-Luc cells stably express luciferase. A lower expression (total flux) indicates a good intracellular delivery of the siRNA and subsequent signalling of the target luciferase mRNA.

Overall, the results show higher transfection efficiency for the composition rich in L-PPI, compared to the composition rich in L-PEI. Moreover the results show that the composition also works in vivo as well as with modified mRNA and siRNA.

Physicochemical Properties of the Compounds

Zeta Potential

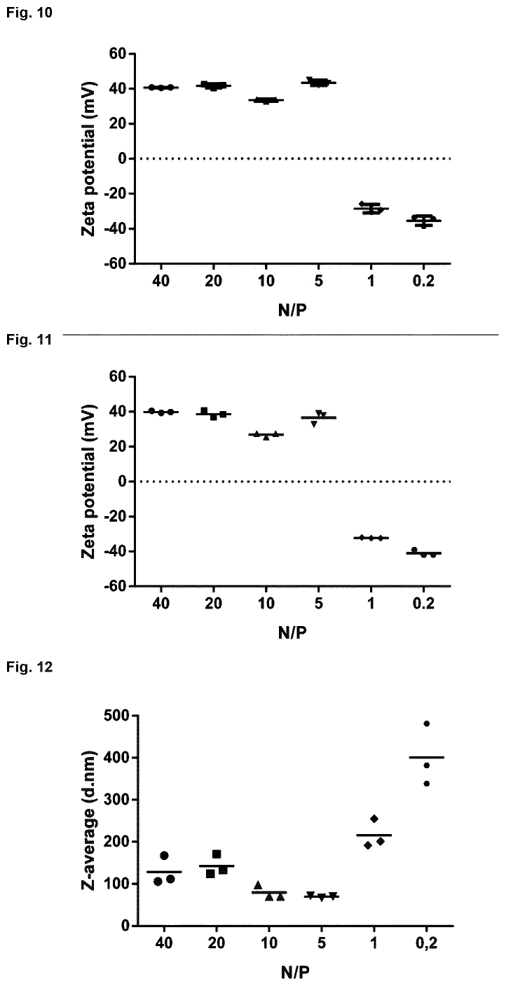

FIG. 10 illustrates measures of zeta potential for compositions comprising self-amplifying mRNA and linear PPI (L-PPI) having DP 250. As illustrated, compositions comprising an N/P ratio of at least 5 have an excellent zeta potential, and are considered stable formulations.

FIG. 11 illustrates measures of Z potential compositions comprising self-amplifying mRNA and a co-polymer of linear PEI and linear PPI (L-PEI/L-PPI) having DP 50/200 (ratio of 1:4). As illustrated, compositions comprising an N/P ratio of at least 5 have an excellent zeta potential, and are considered stable formulations.

Size Measurement

FIG. 12 illustrates size measurements of compositions comprising linear PPI (L-PPI) having DP 250 and replicon RNA (self-amplifying mRNA). Composition having a N/P ratio of 1 or less where shown to have a higher Z-average compared to compositions having a higher N/P ratio. For some applications, a low average diameter of the particles may be beneficial.

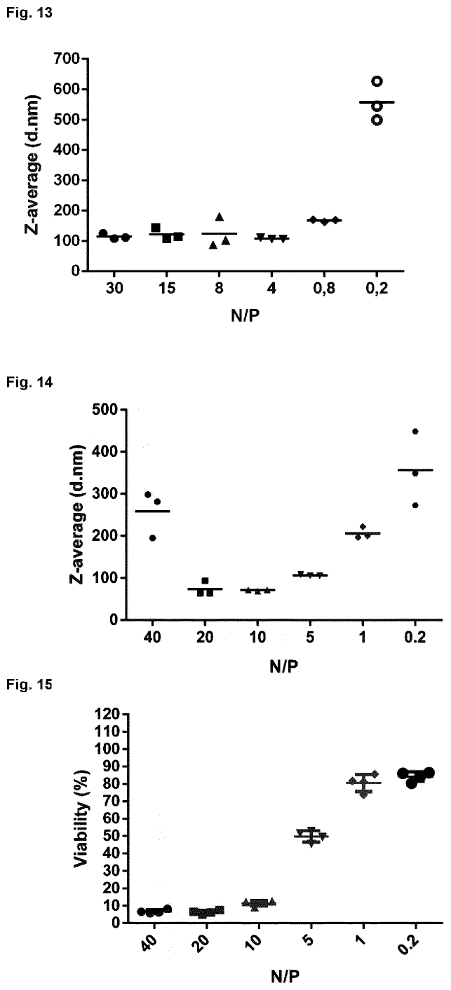

FIG. 13 illustrates size measurements of compositions comprising modified mRNA and linear PPI (L-PPI) having DP 250. Composition having a N/P ratio of 0.2 or less where shown to have a higher Z-average compared to compositions having a higher N/P ratio. For some applications, a low average diameter of the particles may be beneficial.

FIG. 14 illustrates size measurements of compositions comprising self-amplifying mRNA and a co-polymer of linear PEI and linear PPI (L-PEI/L-PPI) having DP 50/200 (ratio of 1:4) and replicon RNA. For co-polymers rich in PPI, a low average diameter of the particles is obtained for compositions having a N/P of 5-20.

Cell Viability

FIG. 15 illustrates the cell viability after 24 h transfection with compositions comprising self-amplifying mRNA and linear PPI (L-PPI) having DP 250. As evident from the figure, the lower the N/P ratio, the lesser the toxicity of the compositions.

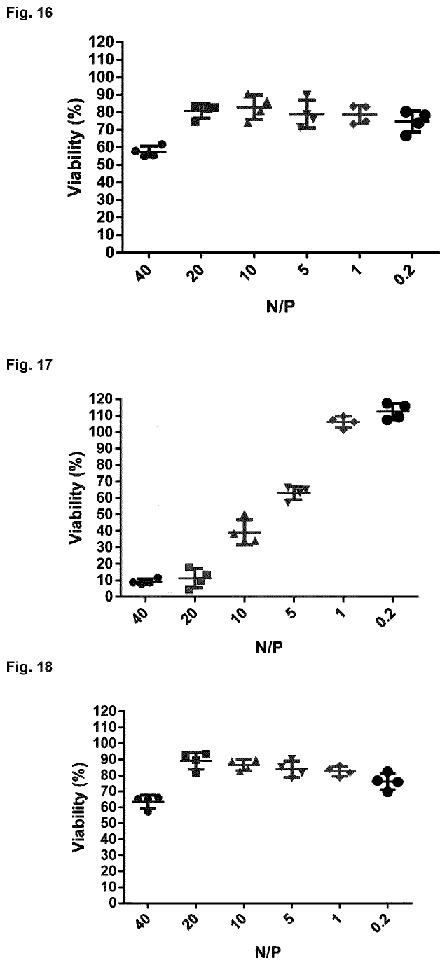

FIG. 16 illustrates the cell viability after 24 h transfection with compositions comprising self-amplifying mRNA and linear PEI (L-PEI) having DP 250. Contrary to the results obtained for PPI, the N/P ratio does not significantly affect the toxicity of compositions comprising high amounts of PEI.

FIG. 17 illustrates the cell viability after 24 h transfection with compositions comprising self-amplifying mRNA and a co-polymer of linear PEI and linear PPI (L-PEI/L-PPI) having DP 50/200 (ratio of 1:4). For the co-polymers, again the lower the N/P ratio, the lesser the toxicity of the compositions.

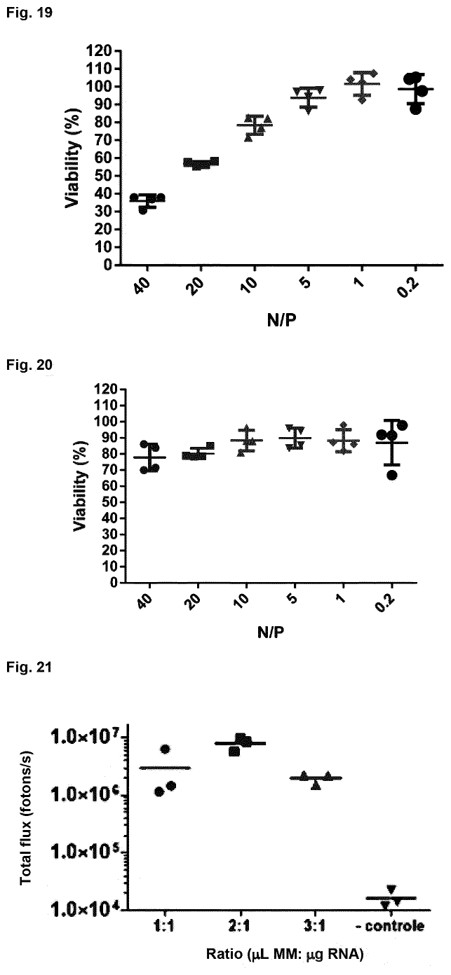

FIG. 18 illustrates the cell viability after 24 h transfection with compositions comprising self-amplifying mRNA and a co-polymer of linear PEI and linear PPI (L-PEI/L-PPI) having DP 200/50 (ratio of 4:1). Contrary to the results obtained for PPI, the N/P ratio does not significantly affect the toxicity of compositions comprising high amounts of PEI.

FIG. 19 illustrates the cell viability after 24 h transfection with compositions comprising self-amplifying mRNA and a co-polymer of linear PEI and linear PPI (L-PEI/L-PPI) having DP 100/150 (ratio of 2:3). For the co-polymers, again the lower the N/P ratio, the lesser the toxicity of the compositions.

FIG. 20 illustrates the cell viability after 24 h transfection with compositions comprising self-amplifying mRNA and a co-polymer of linear PEI and linear PPI (L-PEI/L-PPI) having DP 150/100 (ratio of 3:2). Contrary to the results obtained for PPI, the N/P ratio does not significantly affect the toxicity of compositions comprising high amounts of PEI.

FIG. 21 Illustrates transfection results of in vitro tests of transfection efficiency carried out with lipofectamine MessengerMax (MM), a state-of-the-art transfection agent. Compositions comprising this lipid carrier and nucleic acids with different ratios were prepared. The ratio thereby illustrated is μl MM:μg mRNA. A control solution with only buffer and thus containing no nanocomplexes was also prepared (controle). Typical transfection efficiency observed with MM is between 1×10⁶ and 1×10⁷. As evident from FIGS. 1 and 3, the compositions of the present invention are at least equally efficient, or even better, i.e. reaching transfection efficiencies of between 1×10⁷ and 1×10⁸.

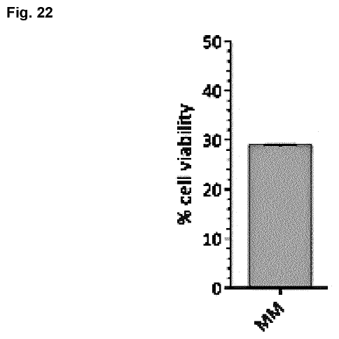

FIG. 22 illustrates the cell viability after 24 h transfection with a composition comprising a lipofectamine MessengerMax (MM) at a ratio of 2:1 (μl MM:μg mRNA). MM was shown to have a cell viability of only about 30%, in contrast and as evident from FIGS. 11 to 16, cell viability of above 50%, even close to 100% can be achieved by the compositions of the present invention. It is important to notice that the cell viability was measured after a 24 h transfection period.

### IN VIVO EXPERIMENTS

In addition, we performed an in vivo transfection experiment in chickens using a composition comprising self-amplifying mRNA encoding luciferase and a co-polymer of linear PEI and linear PPI (L-PEI/L-PPI) having DP 50/200. Bioluminescence images were taken shortly after euthanasia, since the visible bioluminescent signal is often an underestimation of the real signal as the light generating enzymatic conversion of D-luciferin requires ATP is known to show a rapid drop after euthanasia. The result show a clear bioluminescent signal in the transfected chicken compared to non injected control chicken (data not shown).

1. A pharmaceutical composition comprising:
(a) a polypropyleneimine polymer (PPI); and
(b) a nucleic acid, and
characterized in that said PPI has a degree of polymerization from about 20 to 1000, preferably from about 100 to 500, most preferably from about 200 to 300.

2. The pharmaceutical composition according to claim 1, wherein said PPI is linear.

3. The pharmaceutical composition according to anyone of claims 1 or 2 further comprising a polyethyleneimine polymer (PEI).

4. The pharmaceutical composition according to claim 3; wherein said PEI has a degree of polymerization from about 20 to 1000, preferably from about 100 to 500, most preferably from about 200 to 300.

5. The pharmaceutical composition according to anyone of claim 3 or 4, wherein said PEI is linear.

6. The pharmaceutical composition according to anyone of claims 1 to 2, wherein said PPI is in the form of a PPI/PEI co-polymer.

7. The pharmaceutical composition according to claim 6, wherein said co-polymer is a random co-polymer.

8. The pharmaceutical composition according to anyone of claim 6 or 7, wherein the co-polymer has a degree of polymerization from about 20 to 1000, preferably from about 100 to 500, most preferably from about 200 to 300.

9. The pharmaceutical composition according to anyone of claims 3 to 8, wherein the degree of polymerization of said PEI to the degree of polymerization of said PPI is within a range from about 1:1 to 1:500, preferably from about 1:1 to 1:100, most preferably from about 1:2 to 1:10.

10. The pharmaceutical composition according to anyone of claims 1 to 9, wherein the pharmaceutical composition further comprises a lipid.

11. The pharmaceutical composition according to anyone of claims 1 to 10, wherein the nucleic acid is an RNA or DNA molecule; preferably selected from the list comprising mRNA, self-replicating mRNA (replicon), circular mRNA, circular RNA, a mRNA or replicon whose translation can be controlled by an external or internal molecule, non-coding RNA, siRNA, sense RNA, antisense RNA, a ribozyme, an RNA aptamer, an RNA aptazyme, saRNA, pDNA, mini circles, closed linear DNA, genomic DNA, cDNA, either single- and/or double-stranded DNA, and any combination or chemical modified version thereof.

12. A pharmaceutical composition according to anyone of claims 1 to 11; wherein the N/P ratio is less than 40; preferably less than 20; more preferably less than 10.

13. A pharmaceutical composition according to anyone of claims 1 to 12, for use in human or veterinary medicine.

14. A pharmaceutical composition according to anyone of claims 1 to 13, for use in (nucleic acid) vaccination, nucleic acid-based protein therapy, nucleic-acid based protein replacement therapy, gene editing, base editing, cell therapy, immunotherapy, stem cell therapy, regenerative medicine, gene silencing, nucleic acid inhibition or protein inhibition.
